(19) United States
(12) Patent Application Publication
(10) Pub. No.: US 2012/0323022 A1
(43) Pub. Date: Dec. 20, 2012

(54) (2'S)-COLUMBIANETIN ISOLATED FROM CORYDALIS HETEROCARPA AND COMPOSITION CONTAINING THE SAME

(76) Inventors: Se Kwon KIM, Dongnae-gu (KR); Chang Suk Kong, Yeonje-gu (KR); Young Wan Seo, Suyeong-gu (KR); Jung Ae Kim, Nam-gu (KR); Byul Nim Ahn, Busanjin-gu (KR)

(21) Appl. No.: 13/495,112

(22) Filed: Jun. 13, 2012

(30) Foreign Application Priority Data
Jun. 14, 2011 (KR) 10-2011-0057677

(51) Int. Cl. C07D 493/14

(52) U.S. Cl. 549/282

(57) ABSTRACT

The present invention relates to a compound of (2′S)-columbianetin having photo-protective effect isolated from Corydalis heterocarpa and a cosmetic composition containing the same, which has great effects on prevention and treatment in UVB-induced skin damage.

### TECHNICAL FIELD

The present invention relates to a compound of (2′S)-columbianetin having the photo-protective effect isolated from Corydalis heterocarpa and a photo-protective cosmetic composition containing the compound.

### BACKGROUND ART

The skin is the largest organ in the body, which includes the accessory structures such as hair and glands. The skin is made up of three layers, epidermis, dermis and subcutaneous tissue. Among them, epidermis is the outermost layer of the skin, which is continuously influenced by harmful environmental factors such as ultraviolet (UV) irradiation of the sun. Solar UV rays are divided by the wavelength into 3 categories; UVA (400-315 nm), UVB (315-280 nm) and UVC (280-100 nm). Most of the UVC rays cannot pass through the ozone layer and it does not reach the earth. Therefore, its role on skin damage is minimal. Both UVA and UVB portions reaching the earth's surface result in significant damage to the human skin. Especially, middle-wave UVB ray is almost absorbed by epidermis with the strongest energy intensity, thereby, it acts primarily on the epidermal basal cell layer of the skin, keratinocytes, inducing harmful biological effects both directly and indirectly, in particular formation of photoproducts, cell cycle arrest, photoaging, inflammation and photocarcinogenesis. With ozone depletion, UVB percentage of the solar radiation has been gradually increasing over the past several decades. For these important reasons, the destruction of ozone layer is a growing major skin problem.

Molecular responses of skin to UV exposure are initiated by photochemical generation of reactive oxygen species (ROS), which can attack cellular components of skin such as lipids, proteins and DNA. In addition, UV-induced ROS is closely related to expression of matrix metalloproteinases (MMPs). MMPs play the substantial roles in extracellular matrix (ECM) degradation during pathological processes such as arthritis, inflammation, cardiovascular diseases and cancer. Among various types of MMPs, MMP-2 (gelatinase A, 72 kDa) and MMP-9 (gelatinase B, 92 kDa) are capable of degrading epidermal basement membrane. The critical biological feature of UVB irradiated skin is a form of apoptotic cell death of keratinocytes, which is morphologically characterized by several unique cellular changes, such as cell shrinkage, chromatin condensation, genomic DNA fragmentation and membrane blebbing.

Corydalis heterocarpa (Siebold et Zuccarini) is classified as a biennial herb with spikes yellow flowers, which is mainly growing on the salt marshes throughout shores or seashores of South Korea. It is a kind of salt-tolerant plant and has been used as a folk medicine for curing contractions and spasm.

Korean Patent Publication No. 10-2011-001538 discloses flowers extract having anti-oxidation and whitening effects such as Corydalis incisa and Corydalis turtschaminovii. A cosmetic composition for whitening of the skin comprising the extract of corydalis yanhusuo as active ingredient is disclosed in Korean Patent Publication No. 10-2009-0120090. However, little study is available in the literature about the antioxidant and anti-inflammatory activities of components isolated from Corydalis heterocarpa up to now.

### BRIEF DESCRIPTION OF THE DRAWINGS

FIG. 1 shows cell viability and morphological changes in HaCaT cells exposed to different irradiation intensities of UVB. (A) MTT assay. (B) LDH release assay. (C) Microscopic image of morphological changes in HaCaT cells. ^{a-d} Means with the different letters are significantly different (p<0.05) by Ducan's multiple range test. Blank: no UVB exposure, Control: cells lysed by addition of Triton X-100.

FIG. 2 shows effect of (2′S)-columbianetin on cytotoxicity and morphological change of HaCaT cells exposed to 40 mJ/cm² of UVB irradiation. (A) MTT assay. (B) LDH release assay. Blank: no UVB exposure, Control: cells lysed by the addition of Triton X-100. (C) Microscopic image of morphological changes in HaCaT cells in response to UVB irradiation.

FIG. 3 shows effect of (2′S)-columbianetin on intracellular ROS generation induced by UVB irradiation. ^{a-d} Means with the different letters are significantly different (p<0.05) by Ducan's multiple range test. Blank: no UVB exposure.

FIG. 4 shows effect of (2′S)-columbianetin on DNA oxidative damage induced by UVB irradiation.

FIG. 5 shows effect of (2′S)-columbianetin on gene and protein expression levels of MMP-2, MMP-9, TIMP-1 and TIMP-2 in HaCaT cells exposed to 40 mJ/cm² of UVB. The expression levels of MMPs and TIMPs were detected using RT-PCR (A) and, Western blot (B) analysis. GAPDH and β-actin were used as an internal standard.

FIG. 6 shows effect of (2′S)-columbianetin on phosphorylation of MAPK (A) and ASK-1 (B) activation in UVB-exposed HaCaT cells.

FIG. 7 shows effect of (2′S)-columbianetin on AP-1 (A) and phosphorylation of p53 and Bax (B) activation in UVB-exposure HaCaT cells.

### DESCRIPTION OF THE PREFERRED EMBODIMENT

### Technical Problem

The object of the present invention is to provide a compound of (2′S)-columbianetin having the photo-protective effect isolated from Corydalis heterocarpa and a photo-protective cosmetic composition containing the compound.

### Technical Solution

The above object of the present invention was achieved by isolating (2′S)-columbianetin from Corydalis heterocarpa, and then determining the photo-protective effect of (2′S)-columbianetin to measure cytotoxicity, LDH release levels, ROS generation, DNA damage and MMP expression in UVB-exposed cells, and finally elucidating the molecular mechanisms by the signaling pathways of MAPK and AP-1 responsible for the protective effect of (2′S)-columbianetin.

### Advantageous Effects

Through the identification of the photo-protective effect of the compound derived from Corydalis heterocarpa that has not been reported to date and the provision of a photo-protective cosmetic composition containing the compound, the present invention provides a highly effective cosmetic composition having the photo-protective efficacy, antioxidation, and anti-aging for the human skin.

### BEST MODE FOR THE INVENTION

The present invention provides a compound of (2′S)-columbianetin isolated from Corydalis heterocarpa, having the following formula 1:

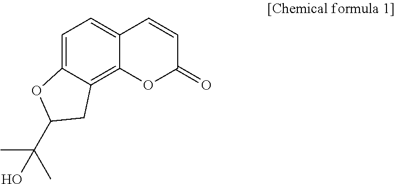

The present invention provides a photo-protective or an anti-aging cosmetic composition containing the above (2′S)-columbianetin.

The cosmetic composition in the present invention can include carrier and conventional supplement such as antioxidant, stabilizer, solubilizer, vitamin, pigment, and perfume.

The cosmetic composition of the present invention can be prepared as any formulation by the well-known method in the art, for example, solution, suspension, emulsion, paste, gel, cream, lotion, powder, soap, surfactant-containing cleansing, oil, powdered foundation, emulsion foundation, a wax foundation, spray, etc, however is not limited thereto. More particularly, the formulation can be prepared as skin softener, skin toner, nutrition lotion, nutrient cream, massage cream, essence, eye cream, cleansing cream, cleansing foam, cleansing water, pack, spray or powder.

In case a composition of the present invention is paste, cream or gel, a carrier component can include animal oil, vegetable oil, wax, paraffin, starch, traganth, cellulose derivative, polyethylene glycol, silicone, bentonite, silica, talc, zinc oxide, etc.

In case a composition of the present invention is powder or spray, the carrier component can include lactose, talc, silica, aluminum hydroxide, calcium silicate or polyamide powder, and spray can additionally comprise propellant such as chlorofluorohydrocarbon, propane/butane or dimethyl ether.

In case a composition of the present invention is solution or emulsion, the carrier component includes solvent, solubilizer or emulsion agent.

In case a composition of the present invention is suspension, the carrier component can contain liquid diluents such as water, ethanol or propylene glycol, suspending agents such as polyoxyethylene sorbitol esters, microcrystalline cellulose, and so on.

In case a formulation of the present invention is surfactant-containing cleansing, the carrier component can comprise aliphatic alcohol sulfate, aliphatic alcohol ether sulfate, etc.

Hereinafter, the present invention will be described by the following examples in more detail. However, the purpose of these examples is only to illustrate the present invention, but not to limit the scope of the invention thereto in any way.

Data were expressed as mean±SD (n=3) and analyzed using the analysis of variance (ANOVA) procedure of Statistical Analysis System (SAS v9.1, SAS Institute Inc., Cary, N.C., USA). Significant differences between treatment means were determined using Duncan's multiple range tests at the p<0.05 level.

### Example 1

### Plant Materials and Isolation of (2′S)-columbianetin

Whole plants of C. heterocarpa were collected in July, 2003 at Muando, Jeollanamdo, Korea. The samples were air-dried, chopped into small pieces, and extracted for 2 days with MeOH (3 L×2) and CH₂Cl₂ (3 L×2), respectively. The combined crude extracts (41.1 g) were evaporated under reduced pressure and partitioned to afford the n-hexane (7.3 g), 85% aqueous (aq.) MeOH (12.0 g), n-BuOH (4.3 g) and water (20.0 g). (2′S)-columbianetin was isolated from a portion of the 85% aq. MeOH fraction.

Six types of fractions were obtained from the above 85% MeOH fraction by using reverse phase chromatography (C₁₈ reversed-phase vacuum flash chromatography). Among them, from the isolate of the fraction No. 1 using the solvent of CH₂Cl₂ containing 10% MeOH on Si gel, (2′S)-columbianetin (658.2 mg) was obtained as follows:

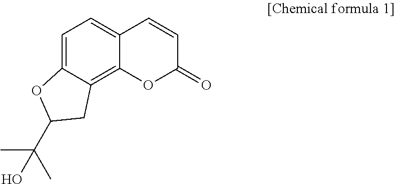

(2′S)-columbianetin Amorphous white solid, mp. 160-163° C.; [α]₂₅^{D}+264° (c 1.1, MeOH); HREI-MS m/z 246.0892 (calcd. for C₁₄H₁₄O₄, 246.0892); ¹H NMR (300 MHz, CD₃OD) δ: 7.82 (¹H, d, J=9.4 Hz, H-4), 7.36 (1H, d, J=8.3 Hz, H-5), 6.76 (1H, d, J=8.3 Hz, H-6), 6.15 (1H, d, J=9.4 Hz, H-3), 4.79 (1H, t, J=9.0 Hz, H-2′), 3.30 (2H, d, J=9.0 Hz, H-1′), 1.30 (3H, s, H-4′/-5′), 1.25 (3H, s, H-4′-5′); ¹³C NMR (75 MHz, CD₃OD) d: 165.5 (C-7), 163.0 (C-2), 152.3 (C-9), 146.1 (C-4), 130.2 (C-5), 115.0 (C-10), 114.2 (C-8), 112.1 (C-3), 107.8 (C-6), 92.5 (C-2′), 72.3 (C-3′), 28.1 (C-1′), 25.4 (C-4′/-5′), 25.3 (C-4′/-5′).

### Example 2

### Cell Culture

Human keratinocyte (HaCaT, Korean Cell Line Bank, Seoul, Korea) cells were grown in Dulbecco's modified Eagle medium (DMEM, Gibco-BRL, Gaithersbrug, Md., USA) containing 10% fetal bovine serum (FBS), 2 mM glutamine and 100 μg/mL penicillin-streptomycin (Gibco-BRL, Gaithersbrug, Md., USA) at 37° C. humidified atmosphere of 5% CO₂. Cells were sub-cultured to about 90-95% confluence by detaching with trypsin-EDTA solution.

### Example 3

### Determination of Optimal UVB Irradiation Level

In order to determine the optimum level of UVB irradiation intensity, the cells were incubated at a density of 1×10⁵ cells/well in 24-well plate with DMEM containing 10% FBS, 2 mM glutamine and 100 μg/mL penicillin-streptomycin at 37° C. humidified atmosphere of 5% CO₂. After incubation for 24 h, the cells were exposed to UVB energy at a range of 10-3000 mJ/cm² (312 nm UVB light source, Bio-Sun lamp, Vilber Lourmat, Marine, France) in 200 μL of phosphate buffered saline (PBS) in each well. After irradiation, the cells were placed in serum-free DMEM for 6, 12, 24 and 48 h.

### Example 4

### Cell Cytotoxicity Determination by MTT and LDH Assays

### Example 4-1

### MTT Assay

The viability levels of keratinocyte cells were determined by the ability of mitochondria to convert 3-(4,5-dimethylthiazol-2-yl)-2,5-diphenyl tetrazolium bromide (MTT) to insoluble formazan product. Briefly, cells were grown in 96-well plates at a density of 1×10⁴ cells/well. After incubation for 24 h, the cells were stimulated by UVB irradiation (40 mJ/cm²) and incubated for 24 h in the absence or presence of (2′S)-columbianetin at 37° C. under a humidified atmosphere of 5% CO₂. The supernatant medium was removed and 100 μL of 1 mg/mL MTT reagent was added to each well, followed by incubation for 4 h. After removal of unconverted MTT, the amount of formazan in the cells was determined by adding DMSO (dimethyl sulfoxide) to wells and measuring optical density (OD) at 540 nm using a microplate reader (Tacan Austria GmbH, Salzburg, Austria). Relative cell viability was calculated as the percentage of viability of untreated cells that were considered the control.

### Example 4-2

### LDH Assay

Cell injury was measured by quantifying the amount of lactate dehydrogenase (LDH)-release using a commercial LDH cytotoxicity detection kit (TaKaRa Biomedicals, Tokyo, Japan). Cells were exposed according to the experiment design. The conditioned medium of the UVB-exposed cells was collected for LDH measurement. The substrate mix solution was added to aliquots of the supernatant medium at 1:1 ratio and incubated at 37° C. for 30 min under light protected conditions. Then, the absorbance was determined at 490 nm immediately after the addition of 1N HCl stop solution (final concentration, 0.2 N) using a microplate reader (Tacan Austria GmbH, Salzburg, Austria). Control was prepared with 0.1% (w/v) Triton X-100, which was defined as 100% LDH release.

### Example 4-3

### Determination of UVB Irradiation Level for Cell Cytotoxicity

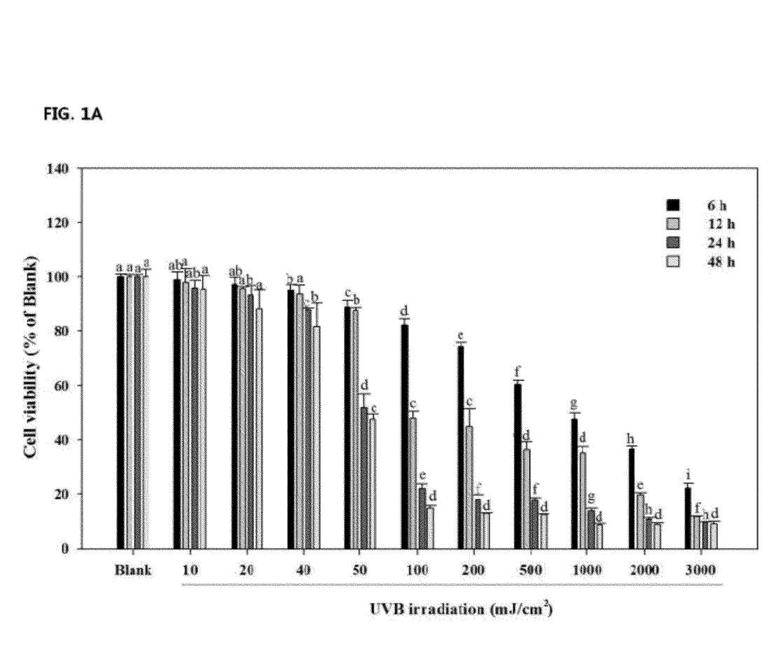

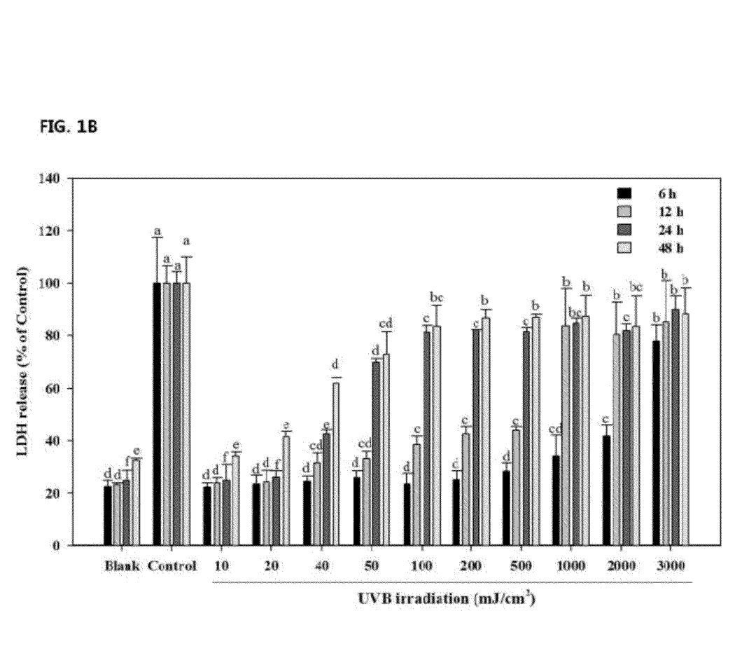

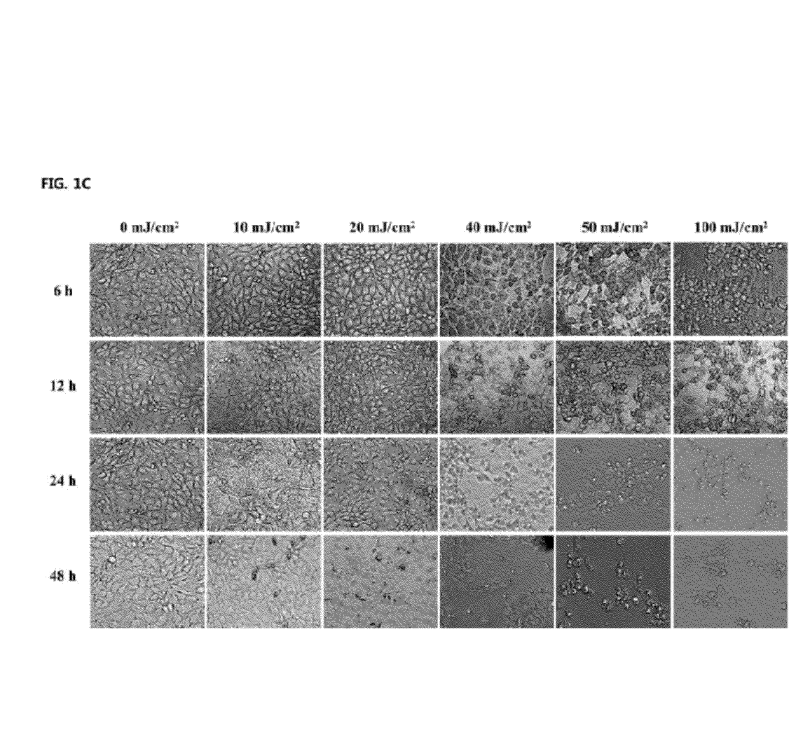

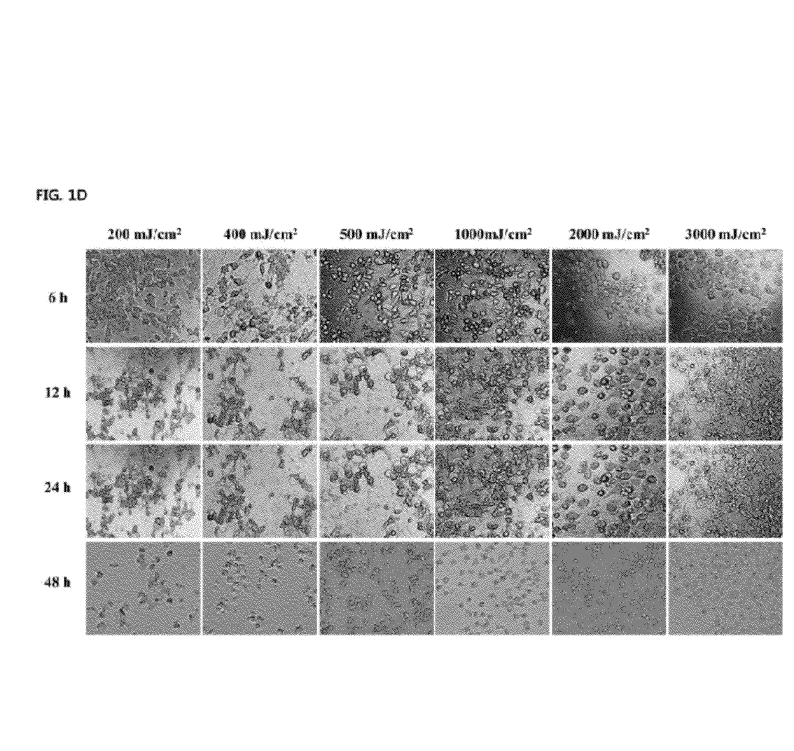

To determine the appropriate energy level of UVB irradiation, the cytotoxicity of keratinocytes cultured from the above was determined by comparing the data obtained from MTT and LDH leakage for 6, 12, 24 and 48 h after UVB irradiation from 10 to 3000 mJ/cm². Exposure of cells to UVB energy induced significant decrease of cell viability (FIG. 1A) and increase of LDH release in a UVB exposure-dependent manner (FIG. 1B). Morphological changes in keratinocyte cells for different incubation periods (6, 12, 24 and 48 h) after UVB exposure of cells in a range of 10-3000 mJ/cm² were compared (FIG. 1C). A larger proportion of cells were swelling and dying in a dose- and time-dependent manner. Consequently, UVB exposure of cells for 24 h at the higher than 40 mJ/cm² dramatically reduced the viability of keratinocyte cells.

### Example 4-4

### Effect of (2′S)-columbianetin on Viability of UVB-Exposed Cells

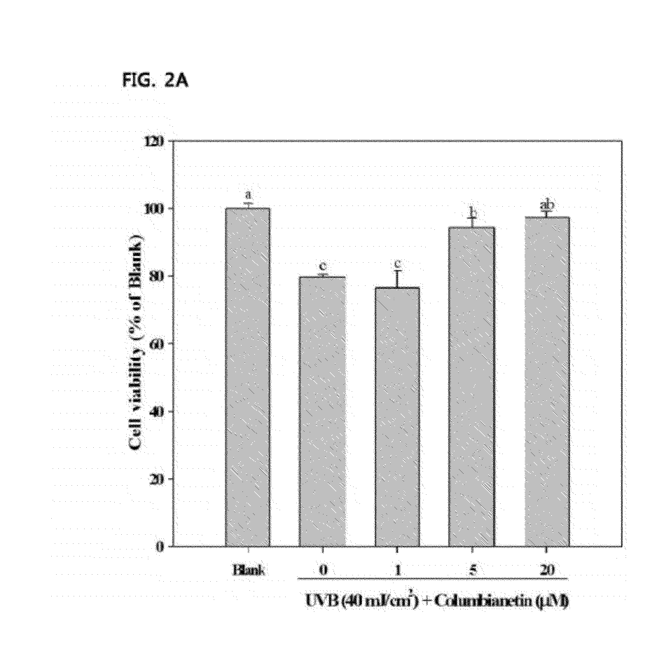

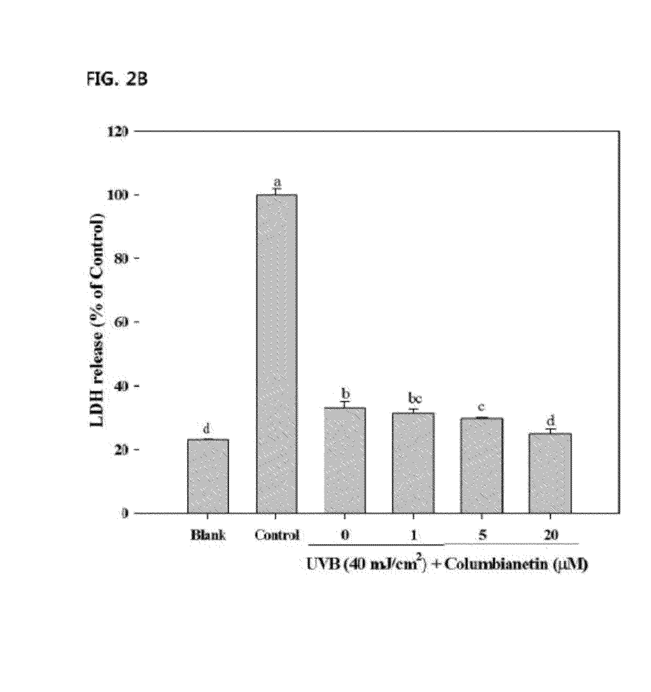

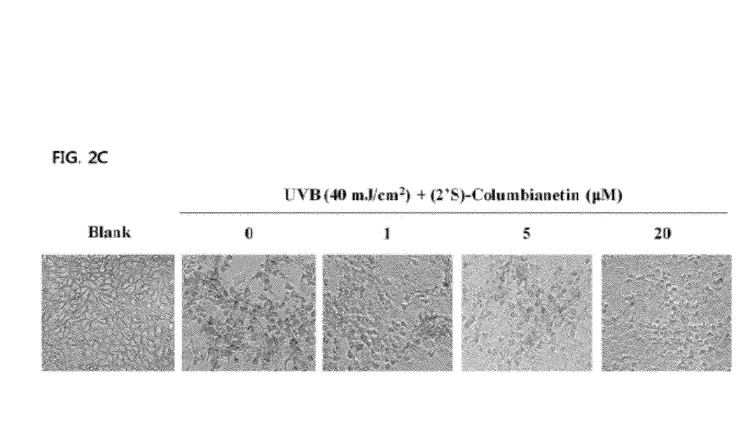

Based on the above results, effects of (2′S)-columbianetin on the viability and injury degree of UVB-exposed keratinocyte cells were examined at incubation for 24 h after UVB exposure of 40 mJ/cm². Exposure of cells to increasing concentrations of compounds substantially increased cell viability in a dose-dependent manner, compared with only UVB-exposed cells (FIG. 2A). LDH release assay revealed that the presence of compounds in UVB-exposed cells substantially decreased the degree of cell injury by UVB exposure in a dose-dependent manner (FIG. 2B). Moreover, the inhibitory effect of (2′S)-columbianetin on cell damage by UVB exposure was confirmed in microscopic image data (FIG. 2C) and the presence of this compound in UVB-exposed cells effectively suppressed the morphological changes in cells due to UV.

### Example 5

### Intracellular Reactive Oxygen Species (ROS) Generation

The levels of intracellular ROS generation were detected using the oxidation sensitive dye 2′,7′-dichlorofluorescin diacetate (DCFH-DA). The cells were grown in a 96-well microplate with fluorescence for 24 h and followed by exposure to UVB (40 mJ/cm²). The exposed cells were treated with (2′S)-columbianetin for 24 h and then they were loaded with 20 μM of DCFH-DA in PBS and incubated for 30 min in the dark at 37° C. under a humidified atmosphere with 5% CO₂. Finally, cells were washed twice with PBS and the fluorescence of DCF in PBS was detected at an excitation wavelength of 485 nm and an emission wavelength of 535 nm using a fluorescence microplate reader (Tacan Austria GmbH, Salzburg, Austria) and the result is shown in FIG. 3.

The increments in DCF fluorescence intensity due to UVB exposure were observed compared with the blank group which was non-UVB exposed. The presence of this compound in UVB-exposed cells significantly reduced DCF fluorescence intensity in a dose-dependent manner, indicating the enhanced ROS scavenging activity against intracellular ROS generated by UVB exposure.

### Example 6

### Genomic DNA Extraction and DNA Oxidation

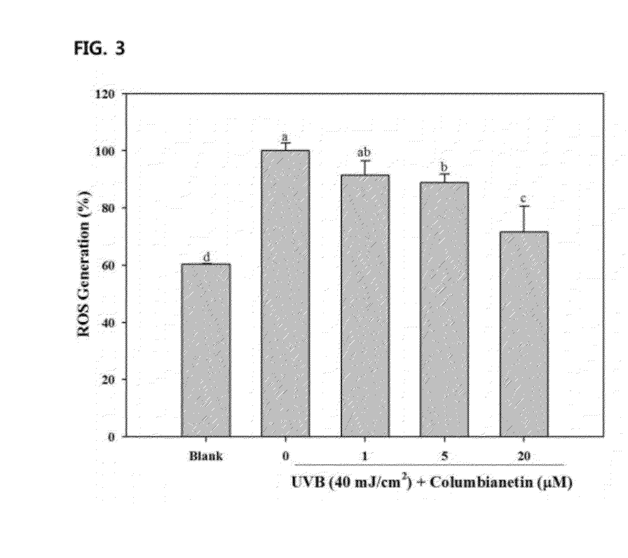

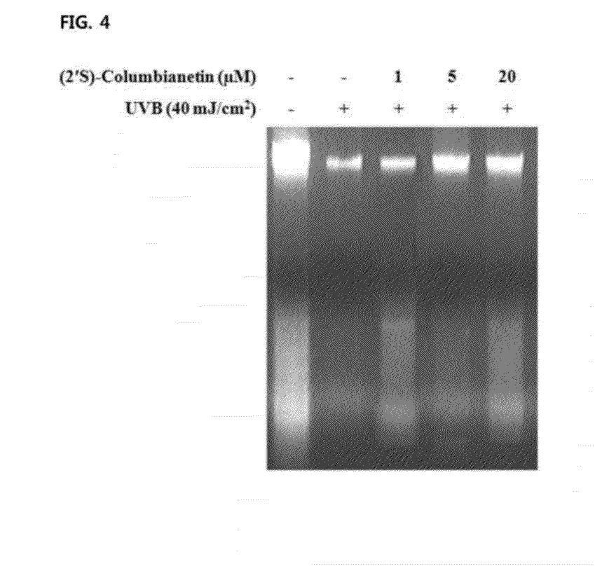

Genomic DNA was isolated from keratinocyte cells using standard phenol/proteinase K procedure with slight modifications (J. Sambrook, D. W. Russell, Molecular Cloning: A Laboratory Manual; 3rd ed. Cold Spring Harbor Laboratory Press: Cold Spring Harbor. 2001). In brief, UVB-stimulated cells in the presence and absence of sample were washed twice with PBS and collected using 1 mL of PBS containing 10 mM EDTA. After centrifugation at 13,400×g for 5 min at 4° C., the deposited cells were resuspended in 410 μL of solution including RNase A (0.5 mg/mL), proteinase K (10 mg/mL), SDS (10%) and NaOAC (0.2 M). After that, this mixture was incubated at 37° C. for 30 min and 55° C. for 1 h. Following incubation, phenol:chloroform:iso-amylalcohol (25:24:1) was added at 1:1 ratio, after which the mixture was centrifuged at 13,400×g for 5 min at 4° C. Then the upper layer was transferred to new eppendorf tube and 100% ice cold ethanol was added at a 1:1.5 ratio followed by incubation at −20° C. for 30 min. After centrifuging at 5,900×g for 5 min at 4° C., the supernatant was carefully removed and the remaining pellet was dissolved in 20 μL of TE buffer (10 mM Tris-HCl, 1 mM EDTA, pH 8.0). Purity of DNA was determined by ratio of absorbance at 260 and 280 nm using spectrophotometric assay. Aliquot (20 μL) of reaction mixture containing about 1 μg of DNA was separated by 1% agarose gel electrophoresis for 10 min at 100 V. The gels were stained by 1 mg/mL of ethidium bromide (EtBr) for 30 min and photographed under UV illumination using AlphaEase® gel image analysis software (Alpha Innotech, San Leandro, Calif., USA) and the result is shown in FIG. 4.

The result confirmed that (2′S)-columbianetin exerted an adequate protective effect on DNA damage due to UVB exposure.

### Example 7

### Effect of (2′S)-columbianetin on the Regulation of UVB-Mediated MMP and TIMP

### Example 7-1

### RT-PCR (Reverse Transcription Polymerase Chain Reaction) Analysis

Total cellular RNA was isolated using Trizol reagent (Invitrogen Co., CA, USA). The 2 μg of isolated RNA was reverse transcribed into cDNA using oligo-(dT) primer (Promega, Madison, Wis., USA). Amplification of target cDNA was amplified using forward and reverse primer sequences: Forward 5′-ATG-GCA-AGT-ACG-GCT-TCT-GT-3′ (SEQ ID NO:1) and reverse 5′-ATA-CTT-CTT-GTC-GCG-GTC-GT-3′ (SEQ ID NO:2) for MMP-2; Forward 5′-CTC-GAA-CTT-TGA-CAG-CGA-CA-3′ (SEQ ID NO:3) and reverse 5′-GCC-ATT-CAC-GTC-GTC-CTT-AT-3′ (SEQ ID NO:4) for MMP-9; Forward 5′-AAT-TCC-GAC-CTC-GTC-ATC-AG-3′ (SEQ ID NO:5) and reverse 5′-TGC-AGT-TTT-CCA-GCA-ATG-AG-3′ (SEQ ID NO:6) for TIMP-1; Forward 5′-TGA-TCC-ACA-CAC-GTT-GGT-CT-3′ (SEQ ID NO:7) and revere 5′-TTT-GAG-TTG-CTT-GCA-GGA-TG-3′ (SEQ ID NO:8) for TIMP-2; Forward 5′-GAG-TCA-ACG-GAT-TTG-GTC-GT-3′ (SEQ ID NO:9) and reverse 5′-GAC-AAG-CTT-CCC-GTT-CTC-AG-3′ (SEQ ID NO:10) for GAPDH. Amplification was carried out at 95° C. for 45 sec, 60° C. for 50 sec and 72° C. for 60 sec for 30 cycles. After amplification step, the extension process proceeded consecutively at 72° C. for 5 min. PCR products were separated on 1% agarose gel for 10 min at 100 V by electrophoresis. Gels were stained with 1 mg/mL of ethidium bromide (EtBr) and photographed by UV illumination using AlphaEase® gel image analysis software (Alpha Innotech., San Leandro, Calif., USA). Finally, the relative band densities were determined using a LAS3000® Luminescent image analyzer (Fujifilm Life Science, Tokyo, Japan).

### Example 7-2

### Western Blot Analysis

Whole cells were lysed in RIPA buffer (Sigma-Aldrich Corp., St. Louis, USA) at 4° C. for 30 min. After centrifugation, total protein amount of cell lysates were determined using Lowry method (BioRad Laboratories, Hercules, Calif.). Aliquot of supernatant containing equal amounts of proteins (10 μg) were electrophoresed on 10% or 12% SDS-polyacrylamide gels, transferred onto a nitrocellulose membrane (Amersham Pharmacia Biotech., England, UK), blocked with 5% bovine serum albumin in TBS containing 0.1% Tween 20 (TBS-T) for at least 1 h, and hybridized with primary antibodies such as MMP-2, MMP-9, TIMP-1, TIMP-2, ERK, JNK, pERK, pJNK, p38, pp 38, ASK1, p-ASK1, c-jun, c-fos, p53, pp 53 and Bax (Santa Cruz Biotechnology Inc., CA, USA). All primary monoclonal antibodies were diluted with TBS-T at a 1:1000 ratio. Bound antibodies were detected by horseradish peroxidase-conjugated secondary antibodies for 1 h at room temperature and the immunoreactive proteins were detected using a chemiluminescent ECL assay kit (Amersham Pharmacia Biosciences, England, UK) according to the manufacturer's instructions. Western blot bands were visualized using a LAS3000® Luminescent image analyzer (Fujifilm Life Science, Tokyo, Japan).

The regulation levels of MMP-2, MMP-9 and tissue inhibitor of metalloproteinase TIMP-1 and TIMP-2 in UVB-exposed cells were determined using RT-PCR and Western blotting (FIGS. 5A and B). Only UVB alone exposed cells exhibited higher MMP-2 and MMP-9 gene expression than UVB non-exposed cells. However, the expression levels of UVB-mediated gelatinase A and B gene and protein were reduced by (2′S)-columbianetin in a dose-dependent manner. Furthermore, changes in the expression levels of TIMP-1 and TIMP-2 in UVB-exposed cells were compared. The reduced TIMP-1 and TIMP-2 expression was dose-dependently enhanced by treatment with this compound.

### Example 8

### Inhibitory Effect of (2′S)-columbianetin on MAPK Activation

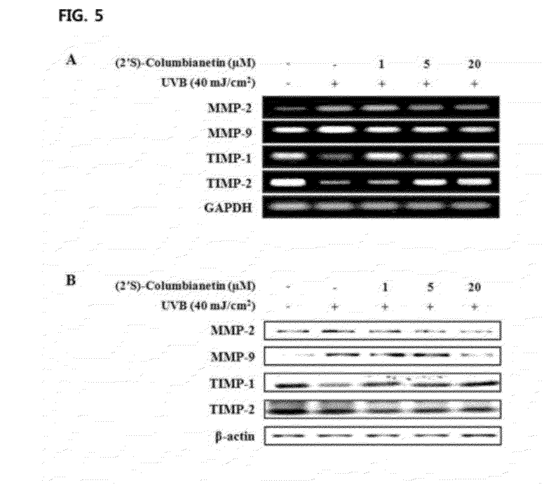

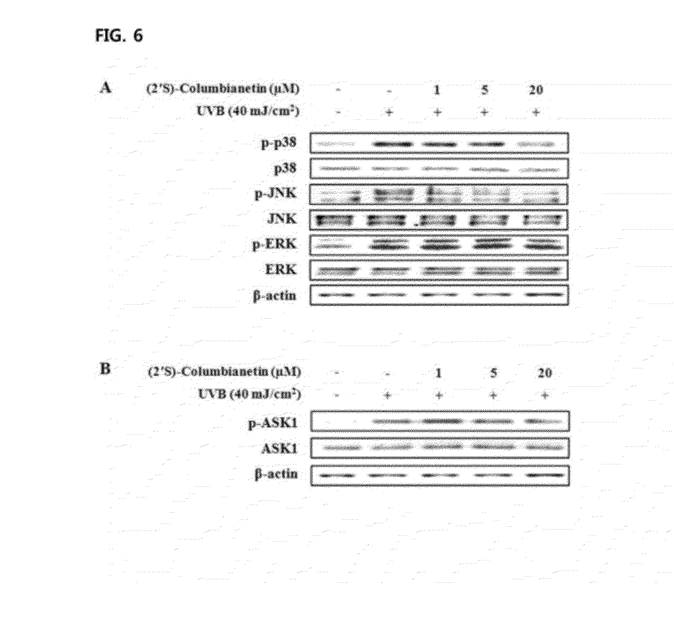

To elucidate the signaling cascades responsible for the protective effect of (2′S)-columbianetin on UVB-exposed cells, apoptosis signal regulation kinase-1 (ASK1) and mitogen-activated protein kinases (MAPK) signaling pathways were studied in UVB-exposed cells (FIG. 6). Effects of (2′S)-columbianetin on the regulations of three major classes of MAPKs, c-Jun N-terminal kinase (JNK), extracellular signal-related kinase (ERK1/2) and p38 MAPK, were investigated in UVB-exposed cells. Although exposure of the cells to UVB irradiation increased the expression of phosphorylated JNK, p38 MAPK and ERK1/2 proteins, the presence of (2′S)-columbianetin in UVB-exposed cells effectively decreased the regulations.

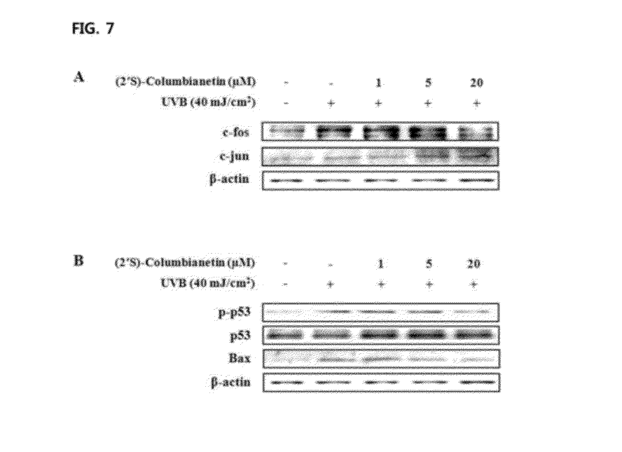

Transcription factor activator protein-1 (AP-1) belongs to the Jun and Fos family. Therefore, we examined the effect of (2′S)-columbianetin on the UVB-induced activation of AP-1. The activated nuclear transcription factor c-fos due to UVB irradiation was significantly down-regulated by (2′S)-columbianetin treatment (FIG. 7A). In addition, upon UVB irradiation of keratinocyte cells, although the expression level of phosphorylated p53 and Bax protein were dramatically increased, the increments were suppressed by (2′S)-columbianetin (FIG. 7B).

What is claimed is:

1. A compound of (2′S)-columbianetin isolated from Corydalis heterocarpa, having the following formula 1:

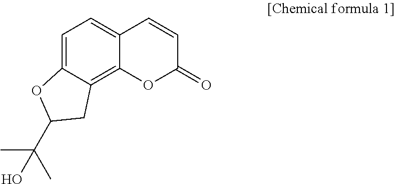

2. A method for preparing (2′S)-columbianetin of claim 1, comprising:
extracting Corydalis heterocarpa with MeOH and CH₂Cl₂;
evaporating the above extract under reduced pressure, followed by fractionating with
MeOH; and
isolating the compound from the said fraction

3. A photo-protective cosmetic composition comprising (2′S)-columbianetin of claim 1.

4. An anti-aging cosmetic composition comprising (2′S)-columbianetin of claim 1.

5. A pharmaceutical composition for preventing or treating skin diseases comprising (2′S)-columbianetin of claim 1.


SEQUENCE LISTING (10 sequences)

SEQ ID NO: 1 - DNA, 20 bp, Homo sapiens
atggcaagtacggcttctgt

SEQ ID NO: 2 - DNA, 20 bp, Homo sapiens
atacttcttgtcgcggtcgt

SEQ ID NO: 3 - DNA, 20 bp, Homo sapiens
ctcgaactttgacagcgaca

SEQ ID NO: 4 - DNA, 20 bp, Homo sapiens
gccattcacgtcgtccttat

SEQ ID NO: 5 - DNA, 20 bp, Homo sapiens
aattccgacctcgtcatcag

SEQ ID NO: 6 - DNA, 20 bp, Homo sapiens
tgcagttttccagcaatgag

SEQ ID NO: 7 - DNA, 20 bp, Homo sapiens
tgatccacacacgttggtct

SEQ ID NO: 8 - DNA, 20 bp, Homo sapiens
tttgagttgcttgcaggatg

SEQ ID NO: 9 - DNA, 20 bp, Homo sapiens
gagtcaacggatttggtcgt

SEQ ID NO: 10 - DNA, 20 bp, Homo sapiens
gacaagcttcccgttctcag